(19) United States
(12) Patent Application Publication
(10) Pub. No.: US 2025/0001021 A1
(43) Pub. Date: Jan. 2, 2025

(54) ANTIMICROBIAL AND STERILIZING DEVICE AND APPLICATIONS OF SAME

(71) Applicant: BOARD OF TRUSTEES OF THE UNIVERSITY OF ARKANSAS, Little Rock, AR (US)

(72) Inventors: Yong Wang, Fayetteville, AR (US); Jingyi Chen, Fayetteville, AR (US); Hugh Churchill, Fayetteville, AR (US); Jin Hu, Fayetteville, AR (US)

(21) Appl. No.: 18/757,897

(22) Filed: Jun. 28, 2024

Related U.S. Application Data
(60) Provisional application No. 63/523,968, filed on Jun. 29, 2023.

(51) Int. Cl. A61L 2/03; A61L 2/232; A61L 2/238

(52) U.S. Cl. CPC A61L 2/03; A61L 2/232; A61L 2/238

(57) ABSTRACT

This invention relates to a antimicrobial and sterilizing device comprising at least one conductive network of nanomaterial(s) formed on a surface of a substrate; and ultra-low electric current applied to the at least one conductive network to generate a self-antimicrobial, self-antifouling, self-sterilizing, energy-efficient, resistant-to-antibiotic-resistance surface on the substrate for killing bacteria, viruses, and harmful microorganisms.

### CROSS-REFERENCE TO RELATED PATENT APPLICATION

This application claims priority to and the benefit of U.S. Provisional Patent Application Ser. No. 63/523,968, filed Jun. 29, 2023, which is incorporated herein in its entirety by reference.

### FIELD OF THE INVENTION

The invention relates generally to the field of materials, and particularly to an antimicrobial and sterilizing device including conductive network layers of nanomaterials powered by microampere electric current for generating self-antimicrobial, self-antifouling, and self-sterilizing surfaces.

### BACKGROUND OF THE INVENTION

The background description provided herein is for the purpose of generally presenting the context of the present invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

After witnessing waves of life-threatening infectious disease outbreaks-including the COVID-19 pandemic and the surge of superbugs accompanying the pandemic-due to viruses, bacteria, and other harmful microorganisms, the importance of developing novel antimicrobial and sterilizing technologies is obvious. During and after the COVID-19 pandemic, it has been a norm to regularly clean, sanitize, and disinfect surfaces in homes and workplaces to prevent the spread of harmful bacteria, viruses, and other microbes. There is an urgent need to develop technologies to produce self-antimicrobial and self-sterilizing surfaces that kills bacteria, viruses, and harmful microorganisms for various applications and in different settings.

### SUMMARY OF THE INVENTION

In one aspect, the invention relates to an antimicrobial and sterilizing device, comprising at least one conductive network of nanomaterial(s) formed on a surface of a substrate; and ultra-low electric current applied to the at least one conductive network to generate a self-antimicrobial, self-antifouling, self-sterilizing, energy-efficient, and/or resistant-to-antibiotic-resistance surface on the substrate for killing bacteria, viruses, and/or harmful microorganisms.

In some embodiments, the nanomaterial has a diameter of about 1-10 nm, a length of about 1-10 μm, and a pore size of about 100 nm-10 μm.

In some embodiments, the nanomaterial comprises a metal, or a conductive material, or a conductive polymer.

In some embodiments, the conductive metal includes gold, silver, copper, iron, nickel, indium, tin, aluminum, magnesium, chromium, or alloys of these materials; and the conductive material include carbon-based materials or combination of metal and carbon based materials.

In some embodiments, the conductive polymers include sulfur-containing polymers such as poly(thiophene)s, poly(3,4-ethylenedioxythiophene), poly(styrene sulfonate), poly(3,4-ethylenedioxythiophene)-poly(styrene sulfonate); and/or nitrogen-containing polymers such as polypyrroles, polycabazoles, polyindoles, polyazepines, polyanilines.

In some embodiments, the at least one conductive network is made of nanowires, nanofibers, and/or nanotubes.

In some embodiments, the at least one conductive network is a random metallic nanowire/nanofiber/nanotube network, wherein the nanowires, nanofibers, and/or nanotubes are randomly aligned.

In some embodiments, the at least one conductive network is a gridded metallic nanowire/nanofiber/nanotube network, wherein the nanowires, nanofibers, and/or nanotubes are gridded.

In some embodiments, the at least one conductive network is a 2D network coated on the surface of the substrate.

In some embodiments, the coating is performed by one or more of coating; roll-milling; brushing; spraying; fabrication; vapor deposition; and on-surface synthesis.

In some embodiments, the ultra-low electric current is about 1 mA or less than 1 mA.

In some embodiments, the ultra-low electric current is generated by a power source of solar cells, home-use batteries, wireless signals, or the like.

In some embodiments, the ultra-low electric current passes through the 2D network, synergizing with the network for antimicrobial applications.

In some embodiments, the ultra-low electric current is applied to the at least one conductive network by direct connection to batteries or solar cells; and/or non-contacting powering methods through wireless signals, RF signals, or light.

These and other aspects of the present invention will become apparent from the following description of the preferred embodiments, taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. The same reference numbers may be used throughout the drawings to refer to the same or like elements in the embodiments.

FIG. 1 shows schematically a device for generating self-antimicrobial, self-antifouling, and self-sterilizing surfaces according to embodiment of the invention. A surface is coated with a 2D network layer made of antimicrobial metallic nanowires or conductive polymers. Ultra-low electric current passes through the 2D network, synergizing with the network for antimicrobial applications.

FIG. 2 shows schematically killing of bacteria on surfaces with random AgNW network and ultra-low electric current measured by colony-forming-unit (CFU) assay, according to embodiment of the invention. Panel (A): Representative TEM image of the random AgNW network. Panel (B): Measured residue CFU of E. coli bacteria on glass surfaces after one day with and without AgNW network and/or electric current of about 300 μA. AgNW with an electric current of about 300 μA killed more than about 99.999% of the bacteria.

FIG. 3 shows schematically killing of bacteria on surfaces with gridded AgNW network and ultra-low electric current of about 1 mA or less measured by microscopy, according to embodiment of the invention. With grid sizes of about 1-2 μm, more than about 99.999% of the bacteria were killed on the AgNW network with electric current of about 1 mA or less.

FIG. 4 shows schematically different grids of AgNW network (about 1-10 nm of diameter, length of about 1-10 μm, pore size of about 100 nm-10 μm), according to embodiments of the invention.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present there between. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this invention, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present invention, and will not be interpreted in an idealized or overly formal sense unless expressly so defined herein.

As used herein, the phrase at least one of A, B, and C should be construed to mean a logical (A or B or C), using a non-exclusive logical OR. As used herein, the term “and/or” includes any and all combinations of one or more of the associated listed items.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

After witnessing waves of life-threatening COVID-19 outbreaks, the importance of developing novel antimicrobial and sterilizing technologies is obvious. In fact, it has been a norm to regularly clean, sanitize, and disinfect surfaces in homes and workplaces to prevent the spread of diseases. Therefore, there is an urgent need to develop technologies to produce better surfaces that are not prone to the spread of bacteria, viruses and other germs.

In view of the foregoing, one of the objectives of this invention is to provide a technology for creating self-antimicrobial and self-sterilizing surfaces for both prevention and disinfection purposes. Particularly, this invention discloses a technology, powered by nanotechnology and ultra-low electric current, to create self-antimicrobial, self-antifouling, self-sterilizing, energy-efficient, resistant-to-antibiotic-resistance surfaces by combining ultra-low electric current and conductive network layers of nanomaterials for various applications and in various settings.

In one aspect, the invention relates to an antimicrobial and sterilizing device, comprising at least one conductive network of nanomaterial(s) formed on a surface of a substrate; and ultra-low electric current applied to the at least one conductive network to generate a self-antimicrobial, self-antifouling, self-sterilizing, energy-efficient, and/or resistant-to-antibiotic-resistance surface on the substrate for killing bacteria, viruses, and/or harmful microorganisms.

In some embodiments, the nanomaterial has a diameter of about 1-10 nm, a length of about 1-10 μm, and a pore size of about 100 nm-10 μm.

In some embodiments, the nanomaterial comprises a metal, or a conductive material, or a conductive polymer.

In some embodiments, the conductive metal includes gold, silver, copper, iron, nickel, indium, tin, aluminum, magnesium, chromium, or alloys of these materials; and the conductive material include carbon-based materials or combination of metal and carbon based materials.

In some embodiments, the conductive polymers include sulfur-containing polymers such as poly(thiophene)s, poly(3,4-ethylenedioxythiophene), poly(styrene sulfonate), poly(3,4-ethylenedioxythiophene)-poly(styrene sulfonate); and/or nitrogen-containing polymers such as polypyrroles, polycabazoles, polyindoles, polyazepines, polyanilines.

In some embodiments, the at least one conductive network is made of nanowires, nanofibers, and/or nanotubes.

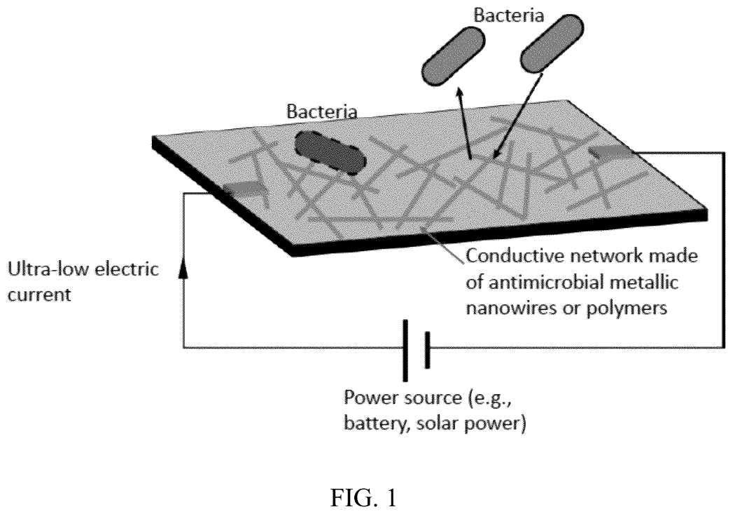

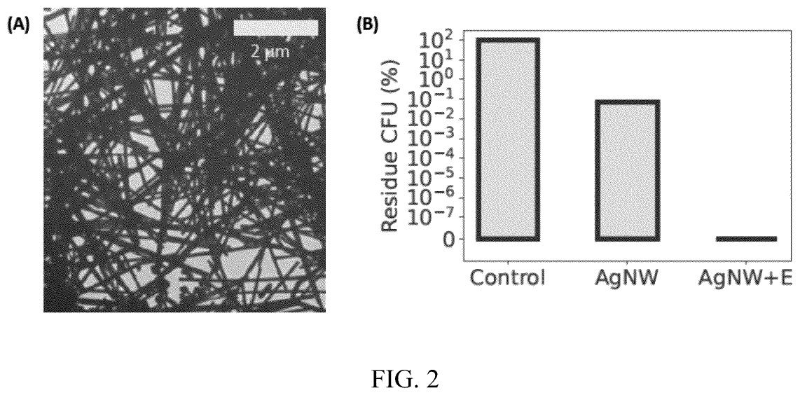

In some embodiments, the at least one conductive network is a random metallic nanowire/nanofiber/nanotube network, wherein the nanowires, nanofibers, and/or nanotubes are randomly aligned, as shown in FIG. 1 and panel (A) of FIG. 2.

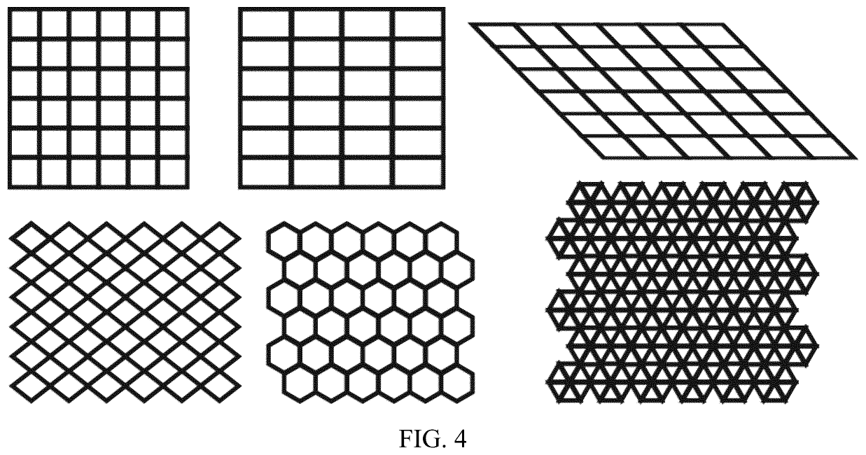

In some embodiments, the at least one conductive network is a gridded metallic nanowire/nanofiber/nanotube network, wherein the nanowires, nanofibers, and/or nanotubes are gridded, as shown in FIG. 4.

In some embodiments, the at least one conductive network is a 2D network coated on the surface of the substrate.

In some embodiments, the coating is performed by one or more of coating; roll-milling; brushing; spraying; fabrication; vapor deposition; and on-surface synthesis.

In some embodiments, the ultra-low electric current is about 1 mA or less than 1 mA. In some embodiments, the ultra-low electric current is generated by a power source of solar cells, home-use batteries, wireless signals, or the like.

In some embodiments, the ultra-low electric current passes through the 2D network, synergizing with the network for antimicrobial applications.

In some embodiments, the ultra-low electric current is applied to the at least one conductive network by direct connection to batteries or solar cells; and/or non-contacting powering methods through wireless signals, RF signals, or light.

As shown in FIG. 1, the device combines ultra-low electric current and conductive network layers of nanomaterials and takes advantage of the synergistic antimicrobial effects from the electric current and the nanomaterial-based network. Specifically, a surface is coated with a 2D network layer made of antimicrobial metallic nanowires or conductive polymers. Ultra-low electric current passes through the 2D network, synergizing with the network for antimicrobial applications.

We have shown that metal (e.g., gold, silver, copper, etc.) nanostructures show antimicrobial activities against bacteria and other microbes. More recently, we reported, for the first time, that bacteria can be killed by ultra-low electric current in the order of microamperes, which can be powered by solar cells, home-use batteries, or wireless signals. However, it has not been invented or reported to synergize the antimicrobial effects of nanomaterials with that of ultra-low electric current for producing self-antimicrobial and self-sterilizing surfaces.

In some embodiments, the exemplary procedure to carry out the invention includes.

Targeted surfaces are coated with a network layer of metal nanowires or conductive polymers by various methods, including but not limited to, coating; roll-milling; brushing; spraying; fabrication; vapor deposition; and/or on-surface synthesis.

Ultra-low current is applied to pass through the conductive network layer of nanomaterials by direct connection to batteries or solar cells or other electric power supplies; and/or non-contacting powering methods through wireless signals, RF signals, or light.

Without intent to limit the scope of the invention, exemplary instruments, apparatus, methods and their related results according to the embodiments of the invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### EXAMPLE 1

### Random Network of Silver Nanowires with Ultra-Low Electric Current

Silver nanowires (AgNWs) were synthesized in solution and then deposited onto glass slides using a Meyer rod coating technique. A representative image of the AgNWs on glass slides is shown in panel (A) of FIG. 2.

E. coli bacteria (as a model system) were introduced to uncoated glass slides (Control), coated glass slides without electric current (Sample: AgNW), and coated glass slides with electric current (Sample: AgNW+E) of about 1 mA or less. The glass slides were incubated at room temperature for overnight (16 hours). Then, surviving bacteria samples on the glass slides were collected by standard environmental sample collection using Q-Swabs and then quantified by standard colony-forming-unit (CFU) assay. Briefly, the collected samples were cultured on LB-agar plates for one night and the number of surviving bacterial colonies (each colony represents a single bacterium in the collected samples) on the plates were counted on the next day.

As shown in panel (B) of FIG. 2, the number of surviving bacteria reduced by 3 orders on the glass slides coated by AgNWs alone. More importantly, the synergic effects of AgNWs+E reduced the number of bacteria to 0 in our measurements (or at least by more than 9 orders). This observation clearly showed that random network of silver nanowires with ultra-low electric current are effective for disinfecting glass surfaces.

It is worthwhile to note that our experiments showed that pre-formed biolfims were disrupted and killed, indicating the effectiveness of ultra-low current with nanomaterials as an antifouling method.

### EXAMPLE 2

### Gridded Network of Silver Nanowires with Ultra-Low Electric Current

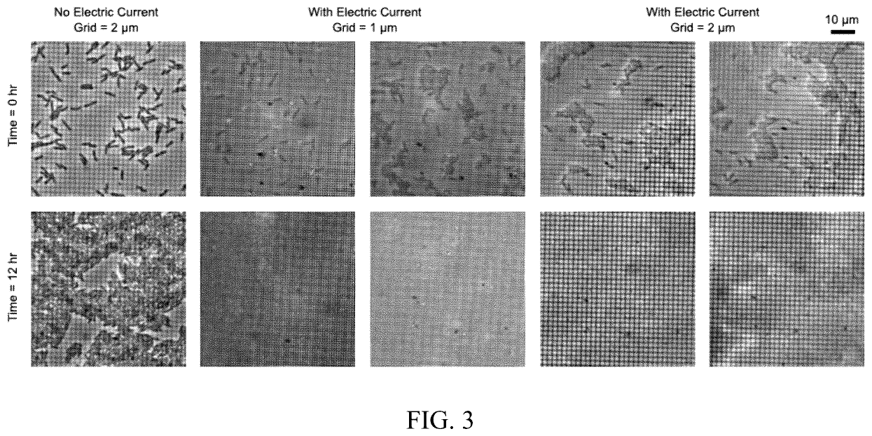

Gridded AgNWs, e.g., shown in FIG. 4, were deposited on glass coverslips using molecular-beam epitaxy (MBE) with different spaces (about 1 μm or about 2 μm), followed by introducing E. coli bacteria to the glass surfaces, without or with electric current below 1 mA. The bacteria were observed under a phase-contrast microscope at the time of introduction. Then the samples were incubated at room temperature for about 12 hours, following by microscopic observation. As shown in FIG. 3, while the gridded AgNW sample without electric current showed the growth of bacteria to some extent, gridded network of AgNWs with electric current killed all the bacteria, consistent with the results from the CFU assay. This observation clearly showed gridded network of silver nanowires with ultra-low electric current are effective for disinfecting glass surfaces and as antifouling coating.

Further improvements of the invention may include, but are not limited to:

Optimizing the coating techniques.

Optimizing the composition of conductive network layer made of nanomaterials.

Optimizing the electric current/power supply passing through the network of nanomaterials.

Demonstrating the invention for various types of surfaces.

Demonstrating the application of the current technology against more microbes/microorganisms.

The novel technology has various advantages, including, but are not limited to: Self-antimicrobial: both ultra-low electric current and the nanomaterials are antimicrobial by themselves. The combination of the two synergistically makes them more effective and efficient for killing bacteria and other microbes.

Self-antifouling: The combination of microampere current and nanomaterials also allows the current technology to disrupt the formation of biofilm by killing the bacteria and other microbe, making the current invention effective for antifouling applications.

Self-sterilizing: electric current is constantly applied to and passing through the conductive nanomaterial-based network for constantly sterilizing the produced surfaces.

Energy-efficient: due to the use of ultra-low electric current, small solar cells, home-use batteries, wireless signal and other methods may be used to power this technology.

Resistance to antibiotic resistance: unlike antibiotics, to which many bacteria have developed resistance, this technology physically damages bacteria and microbes and thus less prone to resistance development of the microbes.

The invention may have, among other things, at least the following applications.

Provide technology to produce long-lasting, self-antimicrobial, self-antifouling, self-sterilizing, self-disinfecting, and energy-efficient surfaces.

Be used as a disinfection technique to treat surfaces.

Be used in food packaging.

Be used in exterior coating of boats, vessels, and ships.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications and various publications, are cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### LIST OF REFERENCES

[1]. Haque M A, Imamura R, Brown G A, Krishnamurthi V R, Niyonshuti I I, Marcelle T, Mathurin L E, Chen J, Wang Y (2017) An experiment-based model quantifying antimicrobial activity of silver nanoparticles on Escherichia coli. RSC Adv., 7 (89): 56173-56182.
[2]. Alqahtany M, Khadka P, Niyonshuti I, Krishnamurthi V R, Sadoon A A, Challapalli S D, Chen J, Wang Y (2019) Nanoscale reorganizations of histone-like nucleoid structuring proteins in Escherichia coli are caused by silver nanoparticles. Nanotechnology, 30 (38): 385101.
[3]. Krishnamurthi V R, Chen J, Wang Y (2019) Silver ions cause oscillation of bacterial length of Escherichia coli. Scientific Reports, 9 (1): 11745.
[4]. Niyonshuti I I, Krishnamurthi V R, Okyere D, Song L, Benamara M, Tong X, Wang Y, Chen J (2020) Polydopamine Surface Coating Synergizes the Antimicrobial Activity of Silver Nanoparticles. ACS Applied Materials & Interfaces, 12 (36): 40067-40077.
[5]. Sadoon A A, Khadka P, Freeland J, Gundampati R K, Manso R H, Ruiz M, Krishnamurthi V R, Thallapuranam S K, Chen J, Wang Y (2020) Silver Ions Caused Faster Diffusive Dynamics of Histone-Like Nucleoid-Structuring Proteins in Live Bacteria. Applied and Environmental Microbiology, 86 (6).
[6]. Wang Y, Wan J, Miron R J, Zhao Y, Zhang Y (2016) Antibacterial properties and mechanisms of gold-silver nanocages. Nanoscale, 8 (21): 11143-11152.
[7]. Durán N, Durán M, Jesus M B de, Seabra A B, Fávaro W J, Nakazato G (2016) Silver nanoparticles: A new view on mechanistic aspects on antimicrobial activity. Nanomedicine: nanotechnology, biology, and medicine, 12 (3): 789-799.
[8]. Krishnamurthi V R, Rogers A, Peifer J, Niyonshuti I I, Chen J, Wang Y (2020) Microampere Electric Current Causes Bacterial Membrane Damage and Two-Way Leakage in a Short Period of Time. Applied and Environmental Microbiology, 86 (16).

What is claimed is:

1. A device for antimicrobial and sterilizing, comprising:
at least one conductive network of a nanomaterial formed on a surface of a substrate; and
ultra-low electric current applied to the at least one conductive network to generate a self-antimicrobial, self-antifouling, self-sterilizing, energy-efficient, and/or resistant-to-antibiotic-resistance surface on the substrate for killing bacteria, viruses, and/or harmful microorganisms.

2. The device of claim 1, wherein the nanomaterial comprises a metal, or a conductive material, or a conductive polymer.

3. The device of claim 2, wherein the conductive metal includes gold, silver, copper, iron, nickel, indium, tin, aluminum, magnesium, chromium, or alloys of these materials; and the conductive material include carbon-based materials or combination of metal and carbon based materials.

4. The device of claim 2, wherein the conductive polymers include sulfur-containing polymers such as poly(thiophene)s, poly(3,4-ethylenedioxythiophene), poly(styrene sulfonate), poly(3,4-ethylenedioxythiophene)-poly(styrene sulfonate); and/or nitrogen-containing polymers such as polypyrroles, polycabazoles, polyindoles, polyazepines, polyanilines.

5. The device of claim 1, wherein the nanomaterial has a diameter of about 1-10 nm, a length of about 1-10 μm, and a pore size of about 100 nm-10 μm.

6. The device of claim 1, wherein the at least one conductive network is made of nanowires, nanofibers, and/or nanotubes.

7. The device of claim 6, wherein the at least one conductive network is a random metallic nanowire/nanofiber/nanotube network, wherein the nanowires, nanofibers, and/or nanotubes are randomly aligned.

8. The device of claim 6, wherein the at least one conductive network is a gridded metallic nanowire/nanofiber/nanotube network, wherein the nanowires, nanofibers, and/or nanotubes are gridded.

9. The device of claim 1, wherein the at least one conductive network is a 2D network coated on the surface of the substrate.

10. The device of claim 9, wherein the coating is performed by one or more of coating, roll-milling, brushing, spraying, fabrication, vapor deposition, and on-surface synthesis.

11. The device of claim 1, wherein the ultra-low electric current is about 1 mA or less than 1 mA.

12. The device of claim 11, wherein the ultra-low electric current is generated by a power source of solar cells, batteries, wireless signals, or the like.

13. The device of claim 1, wherein the ultra-low electric current passes through the 2D network, synergizing with the network for antimicrobial applications.

14. The device of claim 1, wherein the ultra-low current is applied to the at least one conductive network by direct connection to batteries or solar cells; and/or non-contacting powering methods through wireless signals, RF signals, or light.
